(19) United States
(12) Patent Application Publication
(10) Pub. No.: US 2020/0345338 A1
(43) Pub. Date: Nov. 5, 2020

(54) SURGICAL COLLECTION BAG

(71) Applicant: The Second Affiliated Hospital of Soochow University, Suzhou (CN)

(72) Inventor: Qiongzhen REN, Suzhou (CN)

(73) Assignee: THE SECOND AFFILIATED HOSPITAL OF SOOCHOW UNIVERSITY, Suzhou (CN)

(21) Appl. No.: 16/881,061

(22) Filed: May 22, 2020

Related U.S. Application Data
(63) Continuation of application No. PCT/CN2019/085345, filed on Apr. 30, 2019.

(51) Int. Cl. A61B 17/00; A61B 90/92

(52) U.S. Cl. CPC A61B 17/00234; A61B 2017/4216; A61B 2017/00287; A61B 90/92

(57) ABSTRACT

The embodiment of the present disclosure discloses a surgical collection bag. The collection bag includes: a main bag body, three protrusion parts disposed on the main bag body, and a plurality of ports disposed on the three protrusion parts. The ports are used to enable a surgical apparatus to be placed into the main bag body. The minimum diameter of one of the ports is greater than the largest diameter of a tumor. when the tumor is crushed inside a body, a portion of the collection bag in the body is integrally sealed.

### CROSS-REFERENCE TO RELATED APPLICATIONS

This application is a continuation of International Application No. PCT/CN2019/085345, filed on Apr. 30, 2019, which designates the United States of America, the contents of which are incorporated herein by reference in their entirety.

### TECHNICAL FIELD

The present disclosure relates to medical devices, and more particularly to a surgical collection bag.

### BACKGROUND

In a laparoscopic surgery, a laparoscopic lens is inserted into an abdominal cavity, images captured by the laparoscopic lens are transmitted to a subsequent signal processing system through optical fibers by digital imaging technology, and displayed on a dedicated monitor in real-time. According to the images of different angles of the patient's organs displayed on the monitor screen, a doctor analyzes and determines the condition of the patient. After the tumor is cut off under a laparoscopy, the tissue is cut and crushed in the abdominal cavity by a blade that is rapidly rotated through using a crusher, and the diseased tissue is taken out through a small incision of the body surface. Therefore, it is necessary to provide a surgical collection bag, so that the tumor tissue will not spread directly in the abdominal cavity during the crushing process, thus ensuring the safety of surgery.

### SUMMARY

One of the embodiments of the present disclosure may provide a surgical collection bag. The collection bag may include: a main bag body 1, three protrusion parts disposed on the main bag body 1, and a plurality of ports disposed on the three protrusion parts; the port may be used to enable a surgical apparatus to be placed into the main bag body; wherein the minimum diameter of one of the ports may be greater than the largest diameter of a tumor; when the tumor is crushed inside a body 11, a portion of the collection bag in the body 11 may be integrally sealed.

In some embodiments, a first protrusion part 2, a second protrusion part 3, and a third protrusion part 4 may be respectively disposed on the main bag body 1. The main bag body 1 may connect to the outside through the first protrusion part 2, the second protrusion part 3, and the third protrusion part 4. A first port 5, a second port 6, and a third port 7 may be disposed on the first protrusion part 2, the second protrusion part 3, and the third protrusion part 4, respectively.

In some embodiments, when the tumor is crushed inside the body 11, the integrally sealed portion of the collection bag in the body 11 may include: pulling the first port 5, the second port 6, and the third port 7 out of the body, and inserting the main bag body 1, the first protrusion part 2 excluding at least a portion of the first port 5 closed to the main bag body 1, the second protrusion part 3 excluding at least a portion of the second port 6 closed to the main bag body 1, and the third protrusion part 4 excluding at least a portion of the third port 7 closed to the main bag body 1 into the body 11.

In some embodiments, cross-sections of the first port 5, the second port 6, and the third port 7 may be circular.

In some embodiments, the main bag body 1 may be a regular hexagon.

In some embodiments, the second protrusion part 3 may be disposed at an intermediate position of one of the sides of the main bag body 1, and the first protrusion part 1 and the third protrusion part 4 may be respectively disposed on two sides adjacent to the side where the second protrusion part 3 is located.

In some embodiments, the profiles of the first protrusion part 2, the second protrusion part 3, and the third protrusion part 4 may be trapezoidal. The first port 5, the second port 6, and the third port 7 may be respectively located at one end of the first protrusion part 2, the second protrusion part 3, and the third protrusion part 4 away from the main bag body 1.

In some embodiments, a volume of the main bag body 1 may be 2500 ml to 6000 ml.

In some embodiments, lengths of the first protrusion part 2, the second protrusion part 3, and the third protrusion part 4 may be 50 to 100 mm.

In some embodiments, the diameters of the first port 5 and the second port 6 may be 10 mm to 30 mm, the diameter of the third port 7 may be 50 mm to 150 mm, and the third port 7 may be configured to place the tumor into the main bag body 1.

In some embodiments, the third port 7 may be distracted in the body 11 by the distraction device 200.

In some embodiments, identification marks 12 may be disposed respectively on the first protrusion part 2, the second protrusion part 3, and the third protrusion part 4.

In some embodiments, the Identification mark 12 corresponding to different protrusion parts may be used different colored ribbons with different colors.

In some embodiments, the collection bag may be used for a laparoscopic uterine fibroid crushing surgery.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a structural schematic diagram illustrating an exemplary surgical collection bag 100 according to some embodiments of the present disclosure;

FIG. 2 is a structural schematic diagram illustrating an exemplary surgical collection bag 100 which is inflated according to some embodiments of the present disclosure;

FIG. 3 is a structural schematic diagram illustrating an exemplary inflated surgical collection bag 100 in which a surgical apparatus is placed according to some embodiments of the present disclosure;

FIG. 4 is a structural schematic diagram illustrating an exemplary inflated surgical collection bag 100 in an abdominal cavity of a patient, in which a surgical apparatus is placed, according to some embodiments of the present disclosure; and

FIG. 5 is a sectional view of an exemplary distraction device 200 according to some embodiments of the present disclosure.

### DETAILED DESCRIPTION

In the following detailed description, numerous specific details are set forth by way of examples in order to provide a thorough understanding of the relevant disclosure. Obviously, diagrams described below are only some examples or embodiments of the present disclosure. Those having ordinary skills in the art, without further creative efforts, may apply the present disclosure to other similar scenarios according to these diagrams. Unless apparent from the locale or otherwise stated, like reference numerals represent similar structures or operations throughout the several views of the diagrams.

It will be understood that the term “system,” “engine,” “unit,” “module,” and/or “block” used herein are one method to distinguish different components, elements, parts, section or assembly of different levels in ascending order. However, the terms may be displaced by another expression if they may achieve the same purpose.

As used herein, the singular forms “a,” “an,” and “the” may be intended to include the plural forms as well, unless the context clearly indicates otherwise. It will be further understood that the terms “include” and/or “comprise,” when as used herein, specify the presence of operations and/or elements, but do not exclude the presence or addition of one or more other operations and/or elements thereof.

The flowcharts used in the present disclosure illustrate operations that systems implement according to some embodiments of the present disclosure. It is to be expressly understood, the operations of the flowcharts may be implemented not in order. Conversely, the operations may be implemented in inverted order, or simultaneously. Moreover, one or more other operations may be added to the flowcharts. One or more operations may be removed from the flowcharts.

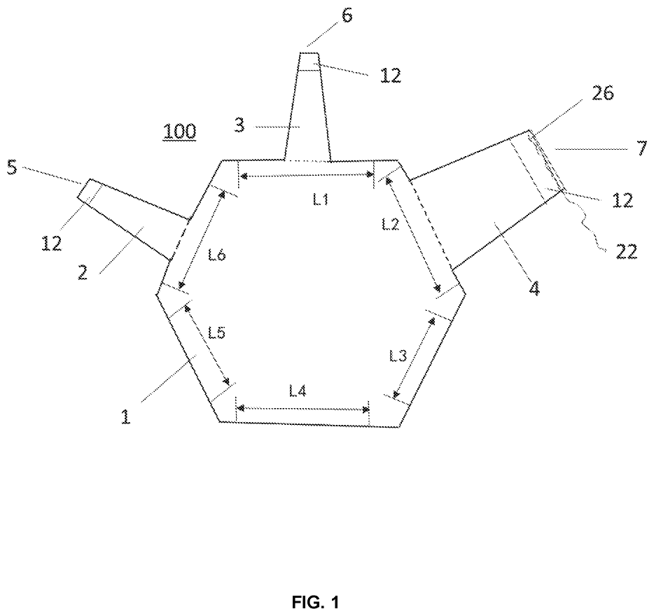

FIG. 1 is a structural schematic diagram illustrating an exemplary surgical collection bag 100 according to some embodiments of the present disclosure.

As shown in FIG. 1, the collection bag 100 may include: a main bag body 1, three protrusion parts disposed on the main bag body 1, and a plurality of ports disposed on the three protrusion parts; the port may be used to enable a surgical apparatus to be placed into the main bag body; where the minimum diameter of one of the ports may be greater than the largest diameter of a tumor; when the tumor is crushed inside a body 11, a portion of the collection bag in the body 11 may be integrally sealed.

In some embodiments, the collection bag 100 may be made of a soft material. The soft material may include but is not limited to, silica gel, polyurethane, poly ternary rubber, medical rubber, or the like, or any combination thereof.

In some embodiments, the collection bag 100 may be used for a laparoscopic surgery for crushing a uterine fibroid.

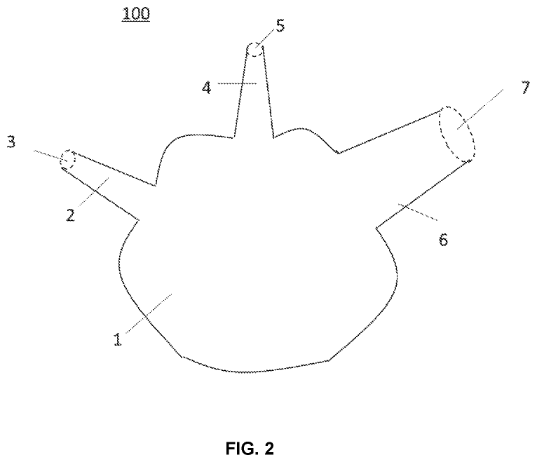

FIG. 2 is a structural schematic diagram illustrating an exemplary surgical collection bag 100 which is inflated according to some embodiments of the present disclosure.

As shown in FIG. 1, the main bag body 1 may be a regular hexagon in an uninflated state. In some embodiments, the main bag body 1 may also have other shapes in the uninflated state, for example, an ellipse, an irregular polygon, or the like. In some embodiments, a volume of the main bag body 1 may be 2500 ml to 6000 ml, for example, the volume of the main bag body 1 after inflation may be preferably 2550 ml to 5500 ml; more preferably, it is 3000 ml to 5000 ml; more preferably, 3500 ml to 4500 ml. As another example, the volume of the main bag body 1 after inflation may be 3550 ml, 3600 ml, 3650 ml, 3700 ml, 4000 ml, 4100 ml, 4200 ml, or the like. In some embodiments, when the main bag body 1 is a regular hexagon without being inflated, the shape may cause the main bag body 1 to closely fit an abdominal wall of a human body in the inflated state, forming an approximately confined space in the main bag body 1, and increasing the operation space.

In some embodiments, a first protrusion part 2, a second protrusion part 3, and a third protrusion part 4 may be respectively disposed on the main bag body 1. The main bag body 1 may connect with the outside through the first protrusion part 2, the second protrusion part 3, and the third protrusion part 4. A first port 5, a second port 6, and a third port 7 may be disposed on the first protrusion part 2, the second protrusion part 3, and the third protrusion part 4, respectively.

In some embodiments, the position of the second protrusion part 3 may be disposed on one side of the main bag body 1, for example, in the middle of the side. The positions of the first protrusion part 2 and the third protrusion part 4 may be respectively disposed on adjacent sides of the side where the second protrusion part 3 is located. For example, as shown in FIG. 1, the sides of the main bag body 1 may be respectively L1 to L6, the position of the second protrusion part 3 may be disposed in the middle of the side L1 of the upper part of the main bag body 1, and the positions of the first protrusion part 2 and the third protrusion part 4 may be respectively disposed on the sides L6 and L2. In some embodiments, the position of the second protrusion part 3 may be set in the middle of the side L1. In some embodiments, the positions of the first protrusion part 2 and the third protrusion part 4 may be symmetrically disposed on the adjacent sides of the side of the second protrusion part 3. For example, the centerlines of the first protrusion part 2 and the third protrusion part 4 may both pass through the midpoint of the side where they are located. In some embodiments, the positions of the first protrusion part 2 and the third protrusion part 4 may be asymmetrically disposed on adjacent sides of the side of the second protrusion part 3. For example, the centerline of the first protrusion part 2 and/or the third protrusion part 4 may not pass through the midpoint of the respective side.

In some embodiments, the profiles of the first protrusion part 2, the second protrusion part 3, and the third protrusion part 4 may be trapezoidal. In some embodiments, the profiles of the first protrusion part 2, the second protrusion part 3, and the third protrusion part 4 may also be other shapes, for example, rectangles, squares, or the like.

In some embodiments, lengths of the first protrusion part 2, the second protrusion part 3, and the third protrusion part 4 may be 50 mm to 100 mm. For example, the lengths of the first protrusion part 2, the second protrusion part 3 and the third protrusion part 4 may preferably range from 55 mm to 95 mm; more preferably, 60 mm to 90 mm; more preferably, 65 mm to 85 mm; more preferably, 70 mm to 80 mm. As another example, the lengths of the first protrusion part 2, the second protrusion part 3, and the third protrusion part 4 may be 72 mm, 74 mm, 76 mm, 78 mm, 80 mm, or the like. A diameter of the first port 5 and the second port 6 may be 10 to 30 mm. For example, the diameters of the first port 5 and the second port 6 may preferably range from 12 mm to 28 mm; more preferably, from 14 mm to 26 mm; more preferably, 16 mm to 24 mm; more preferably, 18 mm to 22 mm. As another example, the diameters of the first port 5 and the second port 6 may be 19 mm, 20 mm, 21 mm, 22 mils, or the like. The diameter of the third port 7 may be 50 mm to 150 mm, for example, the diameter of the third port 7 may preferably range from 55 mm to 145 mm; more preferably, 60 mm to 140 mm; more preferably, 70 mm to 130 mm; more preferably, 80 mm to 120 mm; more preferably, 90 to 110 mm. As another example, the diameter of the third port 7 may be 95 mm, 100 mm, 105 mm, 110 mm, etc.

In some embodiments, the first port 5, the second port 6, and the third port 7 may be respectively located at one end of the first protrusion part 2, the second protrusion part 3, and the third protrusion part 4 away from the main bag body 1. In some embodiments, the first port 5, the second port 6, and the third port 7 may be open, and formed by removing a portion of the smaller ends of the first protrusion part 2, the second protrusion part 3, and the third protrusion part 4. In some embodiments, as shown in FIG. 1, the cross-sections of the first port 5, the second port 6, and the third port 7 in the uninflated state may be straight lines. In some embodiments, as shown in FIG. 2, the cross-sections of the first port 5, the second port 6 and the third port 7 in the inflated state may be circular, which facilitates the placement of a sheath and the surgical apparatus, as well as facilitating the entry and exit of the sheath and the surgical apparatus, and easy to operate.

In some embodiments, after the main bag body 1 is placed in the body 11, the CO₂ gas may be charged into the main bag body 1 through different ports. For example, the CO₂ gas may be charged into the main bag body 1 via a third port 7, which may be provided by a laparoscopic CO₂ pneumoperitoneum system. In some embodiments, the collection bag 100 may be sterilized and placed in the body 11. As shown in FIG. 2, the shape of the inflated bag 100 is a gourd shape. In some embodiments, the shape may cause the main bag body 1 to closely fit the abdominal wall of the human body in the inflated state, form an approximately confined space in the main bag body 1, and increase the operation space.

In some embodiments, the cross-sections of the first port 5, the second port 6, and the third port 7 may be circular, such that when the main bag body 1 is inflated, the first port 5, the second port 6, and the third port 7 may be circular, which facilitates the placement of the sheath. After inflation, the diameter of the first port 5 and the second port 6 may be 10 mm to 30 mm, and the diameter of the third port 7 may be 50 mm to 150 mm. The third port 7 may be configured to place a tumor. Therefore, the diameter of the third port 7 may be larger than the first port and the second port, and the minimum diameter of the third port 7 may be larger than the maximum diameter of the tumor. In some embodiments, when the maximum diameter of the tumor is 40 mm, the minimum diameter of the third port 7 may be set to be more than 40 mm, for example, the minimum diameter of the third port 7 may be set to 50 mm. In some embodiments, the tumor may include but is not limited to, a uterine fibroid, a dysgerminoma, a malignant granulosa cell tumor, a malignant supporting cell tumor, a malignant stromal cell tumor, a rhabdomyosarcoma, a leiomyosarcoma, a liposarcoma, or the like. In some embodiments, when the tumor is crushed inside the body 11, the portion of the collection bag 100 in the body 11 may be integrally sealed, which may include: pulling the first port 5, the second port 6, and the third port 7 out of the body, and inserting the main bag body 1, the first protrusion part 2 excluding at least a portion of the first port 5 closed to the main bag body 1, the second protrusion part 3 excluding at least a portion of the second port 6 closed to the main bag body 1, and the third protrusion part 4 excluding at least a portion of the third port 7 closed to the main bag body 1 into the body 11.

For example, after the collection bag 100 is placed in the body 11 and the main bag body 1 is inflated in the body 11, the tumor may be placed into the main bag body 1 through the third port 7 in conjunction with the surgical apparatus (e.g., a laparoscope). The first port 5, the second port 6, and the third port 7 may be pulled out of the body by the surgical apparatus (e.g., a forceps or an operating forceps) through abdominal pores predetermined on the body. The surgical apparatus may be then placed into the body 11 through each port, and then the tumor may be crushed in the main bag body 1 by a crushing apparatus. In some embodiments, the first protrusion part 2, the second protrusion part 3, and the third protrusion part 4 may be trapezoidal. The first port 5, the second port 6, and the third port 7 may be respectively located at one end of the first protrusion part 2, the second protrusion part 3, and the third protrusion part 4 away from the main bag body 1.

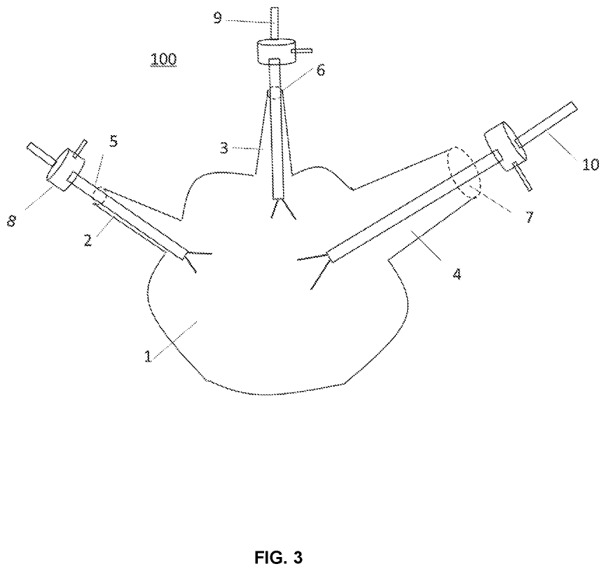

FIG. 3 is a structural schematic diagram illustrating an exemplary inflated surgical collection bag 100 in which a surgical apparatus is placed according to some embodiments of the present disclosure.

In some embodiments, different surgical apparatuses may be placed in the main bag body 1 through the first port 5, the second port 6, and the third port 7. For example, as shown in FIG. 3, a surgical apparatus 8, a surgical apparatus 9, and a surgical apparatus 10 may be respectively placed in the first port 5, the second port 6, and the third port 7. In some embodiments, the first port 5, the second port 6, and the third port 7 may be respectively placed in a first sheath, a second sheath, and a third sheath (not shown in the drawings). Different surgical apparatuses may be placed in the first sheath, the second sheath, and the third sheath. For example, the surgical apparatus 8 may be placed in the first sheath, the surgical apparatus 9 may be placed in the second sheath, and the surgical apparatus 10 may be placed in the third sheath. In some embodiments, the surgical apparatus may include but is not limited to, an operating forceps, a laparoscope, a crushing apparatus, or the like. The shape of the sheath may be an arcuate cannula structure, so that it may be easily placed in the port, and the sheath may be configured to make the surgical apparatus more smoothly placed in the body, preventing the surgical apparatus from puncturing the bag body. In some embodiments, the length of the sheath may be selected according to the length of the protrusion part. In some embodiments, the first port 5 and the second port 6 may be pulled out of the body using the surgical apparatus (e.g., an operating forceps) in conjunction with a laparoscope. In some embodiments, the tumor may be crushed in the main bag body 1 using the surgical apparatus (e.g., a crushing apparatus) in conjunction with the laparoscope. In some embodiments, the operating forceps may be placed into the first protrusion part 2 through the first sheath. In some embodiments, the laparoscope may be placed into the second protrusion part 3 through the second sheath. In some embodiments, the crushing apparatus may be placed into the third protrusion part 4 through the third sheath. In some embodiments, the surgical apparatus may enter the main bag body 1 by being placed in each protrusion part.

In some embodiments, as shown in FIG. 3, the surgical apparatus 8 may be the operating forceps, and the surgical apparatus 9 may be the laparoscope. The operating forceps and the laparoscope may be respectively placed into the first port 5 and the second port 6 through the first sheath and the second sheath. In other embodiments, the surgical apparatus 8 may also be the laparoscope, and the surgical apparatus 9 may also be the operating forceps.

In some embodiments, the tumor may be placed in the main bag body 1 through the third port 7. For example, after the main bag body 1 is inflated, the surgical apparatus may be placed into the body 11 through each port, and the tumor may be placed into the main bag body 1 through the third port 7 in conjunction with the surgical apparatus (e.g., a laparoscopy). The tumor may then be crushed in the main bag body 1 by a crushing apparatus. As shown in FIG. 3, the surgical apparatus 10 may be a crushing apparatus.

In some embodiments, differentiated identification marks 12 may be respectively disposed on the first port 5, the second port 6, and the third port 7. In some embodiments, the identification marks 12 corresponding to different protrusion parts may be used different colored ribbons with different colors. In some embodiments, as shown in FIG. 1, the identification marks 12 may be respectively disposed on the first port 5, the second port 6, and the third port 7 for distinguishing different protrusion parts. In some embodiments, the identification marks 12 may be used different colored ribbons with different colors for different protrusion parts, so that different protrusion parts can be identified by the identification marks 12 during the operation to facilitate the operation. On the other hand, it may be also convenient to pull the different protrusion parts out of the body 11 with the operating forceps. In some embodiments, the identification mark 12 on the first port 5 may be red for identifying the first protrusion part 2, corresponding to place the surgical apparatus 8 (e.g., an operating forceps). The identification mark 12 on the second port 6 may be yellow for identifying the second protrusion part 3, corresponding to place the surgical apparatus 9 (e.g., a laparoscopy). The identification mark 12 on the third port 7 may be blue for identifying the third protrusion part 4, corresponding to place the surgical apparatus (e.g., a crushing apparatus). It should be noted that the present disclosure does not specifically limit the color of the logo selection on each protrusion part, and only needs to be able to identify each protrusion part. For example, it may be selected that the color of the identification mark 12 on the first port 5 is green, the color of the identification mark 12 on the second port 6 is blue, and the color of the identification mark 12 on the third port 7 is yellow.

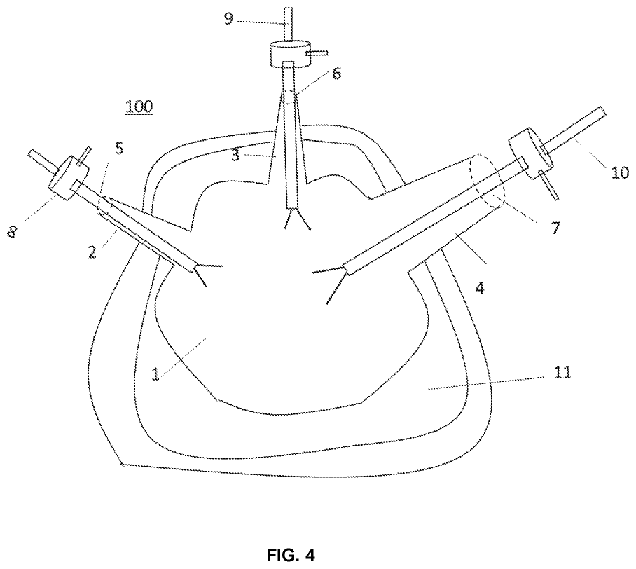

FIG. 4 is a structural schematic diagram illustrating an exemplary inflated surgical collection bag 100 in an abdominal cavity of a patient, in which a surgical apparatus is placed, according to some embodiments of the present disclosure.

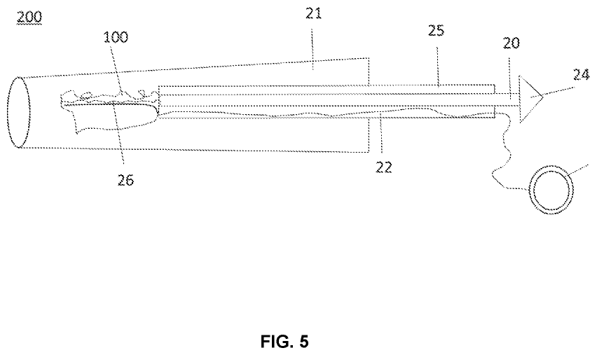

FIG. 5 is a sectional view of an exemplary distraction device 200 according to some embodiments of the present disclosure.

As shown in FIG. 5, the distraction device 200 may include a distraction rod 20, an inner sleeve 25, and an outer sleeve 21. In some embodiments, one end of the distraction rod 20 may pass through the inner sleeve 25, and may be connected to the third port 7. The other end of the inner sleeve 25 may be located outside the inner sleeve 25 and provided with a handle 24 for facilitating the push operation and the pull operation. In some embodiments, one end of the inner sleeve may extend into the outer sleeve 21 and the other end may be located in the outer sleeve 21. A pulling string 22 may pass through the inner sleeve 25. In some embodiments, a collection bag 100 may be placed inside the outer sleeve 21. A portion of the inner sleeve 25 and the distraction rod 20 may also be placed in the outer sleeve 21. In some embodiments, the surgical apparatus (e.g., an operating forceps, a laparoscope, a crushing apparatus, etc.) may be removed from the body and the collection bag may be pulled out of the body through the pulling string 22 on the third port 7.

In some embodiments, the collection bag 100 may be wound up in the distraction device 200 before the use of the distraction device 200. The third port 7 of the collection bag 100 may include a curled side 26 with the pulling string 22 threaded therein. In some embodiments, the pulling string 22 may pass through the curled side 26 and pass through the inner sleeve 25 to the outside of the inner sleeve 25. In order to facilitate the tensioning of the pulling string 22, a pulling tab 23 may be disposed on one end of the pulling string outside the inner sleeve 25 to facilitate the tensioning of the pulling string 22, thereby allowing an operator (e.g., a doctor) to tighten the collection bag 100 in vitro. In some embodiments, both the first port 5 and the second port 6 of the collection bag 100 may include a curled side, and the curled side may be provided with a pulling string. When the collection bag 100 is placed in the body by the distraction device 200, the operator (e.g., a doctor) may pull the pulling string on the first port 5 and the pulling string on the second port 6 out of the body by the surgical apparatus (e.g., a forceps or a surgical forceps) to pull the first protrusion part 2 and the second protrusion part 3 out of the body in the subsequent operation.

In some embodiments, when the distraction device 200 enters the body 11, the distraction rod 20 may be pushed, and the collection bag 100 may extend out of the outer sleeve 21 with the distraction rod 24 and distract the third port 7 of the third protrusion part 4. After the tumor is placed into the collection bag 100 through the third port 7, the distraction device 200 may be withdrawn from the body and separated from the collection bag 100, which makes it convenient for the surgical apparatus 10 to enter the body 11.

It should be noted that the distraction device 200 in the present disclosure may also be other devices known to those skilled in the art that may distract the port of the collection bag. In some embodiments, the distraction device 200 may also be a steel-string. For example, the steel-string may be placed in the curled side of the third port 7, and the third port may be distracted by pulling out the string.

In some embodiments, the collection bag 100 may be sterilized, and then the sterilized collection bag 100 may be placed into the body 11 (e.g., an abdominal cavity) by means of the distraction device (e.g., the distraction device 200 or a steel-string) or other feasible insertion means. And the third port 7 may be pulled out of the body. As shown in FIG. 4, when the collection bag 100 is placed in the body 11 of a patient, gas may be charged into the collection bag 100. In some embodiments, the gas may include CO₂ gas. In some embodiments, after the collection bag 100 is inflated, the wall of the main bag body 1 in the body 11 may abut against the inner wall of the body of the patient, preventing the collection bag 100 from sliding, facilitating the operation during the operation. In some embodiments, the CO₂ gas may be provided by a laparoscopic CO₂ pneumoperitoneum system. In some embodiments, the first port 5 and the second port 6 may be pulled out of the body by a laparoscopy through a forceps or other viable surgical apparatuses. In some embodiments, an operating forceps may be placed in the first port 5, a laparoscope may be placed in the second port 6, and a crushing apparatus may be placed in the third port 7. As shown in FIG. 4, before the crushing apparatus crushes the tumor, at least a portion of the first protrusion part 2, the second protrusion part 3, and the third protrusion part 4 may be outside the human body, thereby realizing that the collection bag 100 in the human body is in a sealed state. In this way, crushed tumor fragments and cell debris will not fall into the human body, causing the diseased tissue to spread in the body and improving the safety of surgery. In some embodiments, the cut tumor may be placed into the main bag body 1 through the third port 7 by operating a forceps or other feasible surgical apparatuses. In some embodiments, the first sheath may be placed on the first port 5 and the operating forceps may be placed in the first sheath. In some embodiments, the second sheath may be placed on the second port 6 and the laparoscope may be placed in the second sheath. In some embodiments, the third sheath may be placed on the third port 7, and the crushing apparatus may be placed in the third sheath. In some embodiments, the crushing apparatus may crush the tumor in the main bag body 1. In some embodiments, the crushing apparatus may crush the tumor into strips. When taken out of the body, the crushing apparatus may be removed from the body together with the crushed tumor.

It should be noted that the above description is provided for the purpose of illustration, and not intended to limit the scope of the present disclosure. It will be understood that those skilled in the art, after understanding the principles of the system, may make various modifications and changes in the form and details of the applications field implementing the collection bag without departing from this principle.

The beneficial effects that the present disclosure embodiment may bring include, but are not limited to: (1) when pulverizing the tumor tissue, the three protrusion parts of the collection bag may be pulled out of the body of the patient so that the crushing process is completed in the collection bag sealed in the body, and the crushed tissue is prevented from splashing in the patient body, and the diseased tissue is further prevented from spreading in the patient body. (2) The collection bag contains three protrusion parts, and different instruments are placed in each protrusion part so that different instruments do not interfere with each other, and the operation is more convenient. It should be noted that different embodiments may have different beneficial effects. In different embodiments, the beneficial effects may be any combination of one or more of the above and may be any other beneficial effects that may be obtained.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment,” “one embodiment,” or “an alternative embodiment” in various portions of this specification are not necessarily all referring to the same embodiment. Furthermore, the particular features, structures, or characteristics may be combined as suitable in one or more embodiments of the present disclosure.

Furthermore, the recited order of processing elements or sequences, or the use of numbers, letters, or other designations, therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose and that the appended claims are not limited to the disclosed embodiments, but, on the contrary, are intended to cover modifications and equivalent arrangements that are within the spirit and scope of the disclosed embodiments.

Similarly, it should be appreciated that in the foregoing description of embodiments of the present disclosure, various features are sometimes grouped in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A collection bag for a surgery, comprising
a main bag body, three protrusion parts disposed on the main bag body, and a plurality of ports disposed on the three protrusion parts, wherein:
the plurality of ports are used to enable a surgical apparatus to be placed into the main bag body;
the minimum diameter of one of the plurality of ports is greater than the largest diameter of a tumor; and
when the tumor is crushed inside a body of a subject, a portion of the collection bag located inside the body is integrally sealed.

2. The collection bag of claim 1, wherein:
the three protrusion parts include a first protrusion part, a second protrusion part, and a third protrusion part, and
the three ports include a first port, a second port, and a third port, wherein
the first protrusion part, the second protrusion part, and the third protrusion part are disposed on the main bag body, respectively, wherein
the main bag body communicates with the outside through the first protrusion part, the second protrusion part, and the third protrusion part, and
a first port, a second port, and a third port are disposed on the first protrusion part, the second protrusion part, and the third protrusion part, respectively.

3. The collection bag of claim 2, wherein when the portion of the collection bag located inside the body is integrally sealed,
the first port, the second port, and the third port are located outside the body, and
the main bag body, the first protrusion part excluding at least a portion of the first port that is close to the main bag body, the second protrusion part excluding at least a portion of the second port that is close to the main bag body, and the third protrusion part excluding at least a portion of the third port that is close to the main bag body are located inside the body.

4. The collection bag of claim 2, wherein:
cross-sections of the first port, the second port, and the third port are circular.

5. The collection bag of claim 1, wherein the main bag body has the shape of a regular hexagon.

6. The collection bag of claim 4, wherein the second protrusion part is disposed at an intermediate position of a side of the main bag body, and the first protrusion part and the third protrusion part are disposed on two sides adjacent to the side where the second protrusion part is located, respectively.

7. The collection bag of claim 2, wherein
profiles of the first protrusion part, the second protrusion part, and the third protrusion part are trapezoidal, and
the first port, the second port, and the third port are located at one end of the first protrusion part that is away from the main bag body, one end of the second protrusion part that is away from the main bag body, and one end of the third protrusion part that is away from the main bag body, respectively.

8. The collection bag of claim 1, wherein a volume of the main bag body is between 2500 ml to 6000 ml.

9. The collection bag of claim 2, wherein lengths of the first protrusion part, the second protrusion part, and the third protrusion part are between 50 mm to 100 mm.

10. The collection bag of claim 2, wherein diameters of the first port and the second port are both between 10 mm to 30 mm, a diameter of the third port is between 50 mm to 150 mm, and the third port is used to enable the tumor to be placed into the main bag body.

11. The collection bag of claim 2, wherein the third port can be distracted inside the body by a distraction device.

12. The collection bag of claim 2, wherein an identification mark is disposed on the first protrusion part, the second protrusion part, and the third protrusion part, respectively.

13. The collection bag of claim 12, wherein the identification marks (12) corresponding to different protrusion parts include different color bands.

14. The collection bag of claim 1, wherein the collection bag is used for a laparoscopic surgery for crushing a uterine fibroid.

15. The collection bag of claim 1, wherein the surgical apparatus includes a crushing apparatus.

16. The collection bag of claim 1, wherein a string is connected to at least one of the plurality of ports.

17. The collection bag of claim 1, wherein the tumor is a uterine fibroid, a dysgerminoma, a granulosa cell tumor, a supporting cell tumor, a stromal tumor, a rhabdomyosarcoma, a leiomyosarcoma, or a liposarcoma.

18. A method for tumor removal, comprising:
placing a collection bag inside a body of a subject;
placing a tumor into the collection bag, wherein the tumor is cut off from the body;
crushing the tumor in the collection bag; and
removing the collection bag from the body, wherein
the collection bag includes a main bag body, a plurality of protrusion parts disposed on the main bag body, and a plurality of ports disposed on the plurality of protrusion parts, wherein
at least one of the plurality of ports is used to enable a surgical apparatus to be placed into the main bag body.

19. The method of claim 18, wherein before crushing the tumor in the collection bag, the method further comprises:
pulling at least one of the plurality of ports out from the body to cause a portion of the collection bag located inside the body to be integrally sealed.

20. The method of claim 18, wherein before crushing the tumor in the collection bag, the method further comprises:
inflating the main bag body.
